(19) United States
(12) Patent Application Publication
(10) Pub. No.: US 2019/0365735 A1
(43) Pub. Date: Dec. 5, 2019

(54) COMPOSITIONS AND METHODS FOR TREATING VARICOSE VEINS

(71) Applicants: Campbell Frederick Mcauley, Los Angeles, CA (US); Taylor Scott Griffith, Alpine, CA (US)

(72) Inventors: Campbell Frederick Mcauley, Los Angeles, CA (US); Taylor Scott Griffith, Alpine, CA (US)

(21) Appl. No.: 15/994,554

(22) Filed: May 31, 2018

(51) Int. Cl. A61K 31/455; A61K 31/685; A61K 31/4166; A61K 36/16; A61K 31/122; A61K 31/593; A61K 31/4415; A61K 33/34; A61K 47/18; A61K 47/02; A61K 47/10; A61K 47/12; A61K 47/14; A61K 47/26; A61K 47/36; A61K 9/00; A61P 9/14

(52) U.S. Cl. CPC A61K 31/455; A61K 2300/00; A61K 31/4166; A61K 36/16; A61K 31/122; A61K 31/593; A61K 31/4415; A61K 33/34; A61K 47/183; A61K 47/02; A61K 47/10; A61K 47/12; A61K 47/14; A61K 47/26; A61K 47/36; A61K 9/0014; A61P 9/14; A61K 31/685

(57) ABSTRACT

A composition for treating varicose veins is provided and has 61.67% Reverse Osmosis Water, 5.5% Glycerin, 5.5% Caprylic Triglycerides, 2.5% Oleic Acid, 2.5% Propylene Glycol, 1.5% Isopropyl Alcohol (or alternatively Ethanol), 1.0% Polysorbate 80, 0.3% Lecithin, 0.8% Xanthan Gum, 0.4% Allantoin, 1.1% Methyl Nicotinate (Nicotinic Acid), 0.3% Pyridoxal-5-Phosphate (P5P), 0.1% Phosphatidylcholine, 5.0% L-Histidine, 5.0% Glycine, 3.0% Gingko Biloba Extract, 0.08% Phytonadione, 0.05% Cholecalciferol, 2.0% Copper Peptides (GHK-Cu), 0.8% Sharon™ Biomix II, 1.0% Herbal Fragrance.

### FIELD OF THE INVENTION

The present invention relates generally to the treatment and correction of venous insufficiency or varicose veins and, more particularly, to a topically applied treatment composition and method to improve varicose veins.

### BACKGROUND OF THE INVENTION

Veins are the blood vessels that return blood to the heart. The veins located in the lower limbs must overcome gravity to perform their job, and are added by the muscles of the calf, which squeeze the veins, pushing blood toward the heart. The lower limbs of the human venous system consist of two main types of veins: the deep veins and the superficial veins.

The deep veins are found underneath the deep fascia of the lower limb, and generally speaking, they accompany and share the name of the major arteries in the lower limb. Often, the deep veins are located within a vascular sheath with the corresponding artery, which helps compress and move blood within the veins. Both types of veins contain a series of venous valves, or one-way flaps of tissue whose job is to prevent blood from flowing backwards, although they are more numerous in the deep veins.

The superficial veins are found in the subcutaneous tissue and are comprised of two major superficial veins—the great saphenous vein and the small saphenous vein. The great saphenous vein is formed by the dorsal venous arch of the foot and the dorsal vein of the great toe. It ascends up the medical side of the leg, passing anteriorly to the medical malleolus of the ankle, and posteriorly to the medical condyle at the knee. As the vein moves up the leg, it receives tributaries from other small superficial veins. The small saphenous vein is formed by the dorsal venous arch of the foot and the dorsal vein of the little toe. It moves up the posterior side of the leg, passing posteriorly to the lateral malleolus, along the lateral border of the calcaneal tendon. Eventually, the superficial veins drain into the deep veins.

However, in some instances, stretching of the veins near the surface of the skin (e.g., the superficial veins) and failure of the venous valves to close properly allows blood to flow in both directions. When a valve is unable to close—also known as an incompetent valve—blood can flow back into the superficial veins; this backwards flow of blood is known as venous reflux, and results in an increased intra-luminal pressure, which veins cannot withstand. Often times, this can lead to twisted, bulging veins, a condition known as varicose veins, or simply, varicosities.

Varicose veins are very common, and affect nearly a third of adults in Western societies. People are more likely to develop varicose veins as they get older, as wear and tear on the veins cause their walls to weaken, thus allowing the vein to enlarge. Some risk factors for varicose veins include a family history of the condition, obesity, pregnancy and standing or sitting for long periods of time.

Individuals with varicose veins may have no symptoms other than the enlarged vein itself, but many do experience discomfort or pain in the vein, sensations such as aching, tightness, burning, itching, or tingling of the legs as well as leg swelling. These symptoms are usually better when first getting out of bed but worsen by the end of the day, after an individual has been sitting or standing for long periods of time. In more severe cases, individuals may have skin changes such as an itchy rash or darkening and thinning of the skin of the legs, which can lead to poorly healing sores. Furthermore, in rare cases the veins may burst and bleed quite dramatically. Varicose veins are diagnosed usually upon a physical examination and in other situations, a vascular ultrasound—a non-invasive test utilizing sound waves—is performed to check for veins with vales that are exhibiting signs of venous reflux.

Because varicose veins are bulging and twisted, blood flow is often restricted and sluggish, which can lead to blood clots. However, unlike blood clots in the deep veins of the legs, clots found in the superficial veins do not usually travel to the lungs, but may cause significant leg swelling, redness, pain, and tenderness at the site of the affected vein. The swelling may have a feeling akin to a firm cord or knot in the leg, and in some patients, prolonged swelling due to varicose veins may lead to skin changes and sores that occur spontaneously without warning or after any minor trauma.

While originally seen as medically unimportant and deserving low priority for treatment, the severe concern and distress caused on a large scale has resulted in a wide variety of treatments evolving rapidly to help address this problem. In most common cases, a diagnosis of varicose veins will be accompanied with a doctor's recommendation to wear elastic compression stockings to control the swelling. Such compression socks are sized specifically to fit the individual's leg and are worn the entire day. While studies have shown that this method may relieve the systems that accompany varicose veins, the stockings do little to make the varicose veins themselves disappear. In addition, patients who are seen as overweight may be encouraged to adopt weight loss methods as a way to deal with their varicose vein diagnosis. Other simple treatment recommendations may include elevating your legs above the heart during the day to decrease swelling, or utilizing a steroid crème or ointment to control inflammation in situations where an individual has developed an itchy rash.

More recently, surgical treatment options have been developed to treat individuals with varicose veins. One example of surgical treatment is known as vein stripping, a technique in which insertions are made in the leg, a long wire is inserted into the vein, and the entire vein is removed through the insertions made. However, this procedure involves general anesthesia, a hospital stay, and a prolonged recovery; this procedure also result in scaring that can be just an unappealing as the appearance of the actual varicose veins. Furthermore, complications such as nerve damage, blood loss, paint, infection, and hematoma occur, and despite the treatment, a high likelihood remained for developing new varicose veins.

Another potential treatment is known as sclerotherapy, a technique which involves injecting a chemical (the “sclerosant”), such as sodium tetradecyl sulfate, into the veins to cause subsequent inflammation and sclerosis of the veins. The sclerosant causes damages in the innermost lining of the vessel, resulting in a clot that blocks the blood circulation in the vein beyond that point, and in turn, forces rerouting of the blood away from the affected veins. While this technically is less surgically intensive than the stripping technique, it often times does not represent a permanent solution.

On this note, advancements have been made in the surgical field relating to the treatment of varicose veins to quell many of the associated drawbacks. For example, U.S. Pat. No. 5,792,168 describes a surgical apparatus to reduce the invasiveness and associated recovery time of the surgical treatments of varicose veins. However, these approaches have their respective limitations and in any event, still require an invasive surgical procedure.

More recently, several patents have discussed treatment of varicose veins with RF energy. For example, U.S. Pat. No. 6,200,312 generally describes a method in which a catheter having an electrode tip is switchably coupled to a source of RF energy, which is then applied to cause located heating and corresponding shrinkage on the adjacent venous tissue. While support for this treatment has increased, this procedure still has many disadvantages as it is quite slow and painful and requires the patient to be anaesthetized along the entire length of the veins to be treated; moreover, this treatment can only be done to a portion of the vein wall (e.g., the portion containing the electrode) and therefore, the partially treated vein may eventually recannularize. This treatment also does not address any tributary veins, as those remain untreated.

As such, a need exists for a non-invasive means, such as a topical composition, which can improve the condition of varicose veins, while avoiding the drawbacks of post and current treatment options.

### SUMMARY OF THE INVENTION

To achieve the forgoing and other aspects and in accordance with the purpose of the invention, a composition for treating varicose veins is presented.

The subject invention relates to a composition that can be used to bring blood flow back to a bulged vein valve.

For the treatment of varicose veins, the composition is formulated to move the vein back to a non-visible location through:

bringing massive blood flow to the bulged vein valve;

allowing the bulged vein vale to return to its normal healthy condition;

causing the bulged vein valve to pull away from the dermis;

increasing the elasticity in the upper skin layers; and

assisting in moving the vein back to a non-visible location.

The skin composition of the present invention is non-irritating and can be used to treat varicose veins and the associated pain and unsightly appearances. It can be generally used anywhere that a bulged vein exists.

This invention is based upon the discovery that these compositions in combination improve the treatment and prevention of varicose veins.

In an embodiment of the present invention, a topical therapeutic composition for treating varicose veins is provided and comprises 61.67% Reverse Osmosis Water, 5.5% Glycerin, 5.5% Caprylic Triglycerides, 2.5% Oleic Acid, 2.5% Propylene Glycol, 1.5% Isopropyl Alcohol (or alternatively Ethanol), 1.0% Polysorbate 80, 0.3% Lecithin, 0.8% Xanthan Gum, 0.4% Allantoin, 1.1% Methyl Nicotinate (Nicotinic Acid), 0.3% Pyridoxal-5-Phosphate (P5P), 0.1% Phosphatidylcholine, 5.0% L-Histidine, 5.0% Glycine, 3.0% Gingko Biloba Extract, 0.08% Phytonadione, 0.05% Cholecalciferol, 2.0% Copper Peptides (GHK-Cu), 0.8% Sharon™ Biomix II, 1.0% Herbal Fragrance.

In an embodiment of the present invention, a method of treating varicose veins is provided which comprises topically applying to the site a composition of 61.67% Reverse Osmosis Water, 5.5% Glycerin, 5.5% Caprylic Triglycerides, 2.5% Oleic Acid, 2.5% Propylene Glycol, 1.5% Isopropyl Alcohol (or alternatively Ethanol), 1.0% Polysorbate 80, 0.3% Lecithin, 0.8% Xanthan Gum, 0.4% Allantoin, 1.1% Methyl Nicotinate (Nicotinic Acid), 0.3% Pyridoxal-5-Phosphate (P5P), 0.1% Phosphatidylcholine, 5.0% L-Histidine, 5.0% Glycine, 3.0% Gingko Biloba Extract, 0.08% Phytonadione, 0.05% Cholecalciferol, 2.0% Copper Peptides (GHK-Cu), 0.8% Sharon™ Biomix II, 1.0% Herbal Fragrance.

Other features, advantages and aspects of the present invention will become more apparent and be more readily understood from the following detailed description, which should be read in conjunction with the accompanying drawings.

### BRIEF DESCRIPTION OF THE DRAWINGS

Other features and advantages of the present invention will become more apparent to a person skilled in the art from the following detailed description in conjunction with the appended drawing in which:

FIG. 1 shows a before and after photograph of the effect of the composition according to one embodiment of the invention.

### DETAILED DESCRIPTION OF THE PREFERRED EMBODIMENTS

The present invention is best understood by reference to the detailed description and examples set forth herein.

Embodiments of the invention are discussed below with reference to the examples. However, those skilled in the art will readily appreciate that the detailed description given herein with respect to these examples is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices and materials are described, although any methods, techniques, devices, or materials similar or equivalent to those described herein may be used in the practice or testing of the present invention.

### Definitions

“Allantoin” as used herein refers to a chemical compound with formula C₄H₆N₄O₃, which is produced from uric acid by urate oxidase and is also a major metabolic intermediate in most organisms, including animals, plants and bacteria.

“Caprylic Triglycerides” as used herein refers to the medium-chain triglycerides who fatty acids have an aliphatic tail of 6-12 carbon atoms. More specifically, in the current embodiment this refers to a trimester derived from coconut oil and glycerin.

“Cholecalciferol” as used herein refers to cholecalciferol, a type of vitamin D made in the skin following ultraviolet-B light exposure.

“Composition” references to that specific composition contemplated in the Summary of the Invention that can be used to treat and prevent varicose veins.

“Copper Peptides” as used herein refers to the naturally occurring copper complex of a glycyl-L-histidyl-L-lysine peptide.

“Derivatives” as used herein refers to structurally similar compounds that exhibit a common activity (e.g., antioxidant) and contain at least one significant, common structural element with the compound from which it is derived, which common structural element provides the common activity.

“Ginkgo Biloba Extract” as used herein refers to an extract made from the dried green leaves from the ginkgo trees.

“Glycerin” as used herein refers to Glycerol as is generally obtained from plant and animal sources where it occurs as triglycerides.

“Glycine” as used herein refers to the amino acid that has a single hydrogen atom as its side chain bearing a chemical formula of NH₂—CH₂—COOH. Glycine is one of the proteinogenic amino acids.

“Herbal Fragrance” as used herein refers to herb plants with strong scents that may be contained within flowers or leaves.

“Isopropyl Alcohol” as used herein refers to an isomer of 1-propanol that contains antibacterial properties.

“Lecithin” as used herein refers to a group of yellow-brownish fatty substances occurring in animal and plant tissues, which are amphiphilic.

“L-Histidine” as used herein refers to an essential amino acid that is one of the 23 proteinogenic amino acids.

“Methyl Nicotinate” as used herein refers to an ester of methyl alcohol and nicotinic acid.

“Oleic Acid” as used herein refers to a fatty acid that occurs naturally in various animal and vegetable fats and oils. In chemical terms, oleic acid is classified as a monounsaturated omega-9 fatty acid, abbreviated with a lipid number of 18:1 cis-9.

“Pharmaceutically-acceptable topical carrier” and equivalent terms refer to an inactive liquid or cream vehicle capable of suspending or dissolving the aromatic aldehyde and having the properties of being nontoxic and non-inflammatory when applied to the skin. Representative carriers include water, oils, both vegetable and mineral, cream bases, lotion bases, ointment bases and the like. These bases include suspending agents, thickeners, penetration enhancers and the like. Their formulation is well known to those in the art of cosmetics and topical pharmaceuticals.

“Phosphatidylcholine” as used herein refers to a class of phospholipids that incorporate choline as a head group.

“Phytonadione” as used herein refers to the group of structurally similar, fat-soluble vitamins the human body requires for complete synthesis of certain proteins that are prerequisites for blood coagulation and which the body also needs for controlling binding of calcium in bones and other tissues.

“Polysorbate 80” as used herein refers to a nonionic surfactant and emulsifier often used in foods and cosmetics, which is derived from polyethoxylated sorbitan and oleic acid.

“Propylene Glycol” as used herein refers to a synthetic organic compound with the chemical formula C₃H₈O₂. Chemically it is classified as a diol and is miscible with a broad range of solvents, including water, acetone, and chloroform.

“Pyridoxal-5-Phosphase” as used herein refers to the active form of vitamin B6 and is a coenzyme in a variety of enzymatic reactions.

“Reverse Osmosis Water” as used herein refers to water that has undergone a water purification process known as reverse osmosis, which uses a semipermeable membrane to remove ions, molecules and large particles from drinking water.

“Sharon™ Biomix II” as used herein refers to biosecur citrus extracts with certain fragrance ingredients such as Phenylpropanol.

“Therapeutically effective dose” is defined as a dose of a composition of this invention which, when applied topically to the skin of a patient afflicted with a dermatologic or other cosmetic or medical condition, or when administered by another route, results in an observable improvement in the patient's condition.

“Topical” refers to a mode of administration and means that a material is administered by being applied to the skin.

“Topically effective” means that a material, when applied to the skin, produces a desired pharmacological result either locally at the place of application or systemically as a result of transdermal passage of an active ingredient in the material.

“Xanthan Gum” as used herein refers to a polysaccharide that is used as an additive.

Now, with reference to the composition for improving the appearance of skin, and in particular, varicose veins, the composition of the present invention is, in some embodiments, applied topically, and may be a cream, gel, ointment, lotion, paste, jelly or other topical.

When applied topically, the composition of the present invention reduces the appearance of varicose veins by bringing massive blood flow to the bulged vein valve, thereby allowing the vein to return to its normal healthy condition, consequently becoming less noticeable. The topically applied composition of the present invention helps reduce the visibility of varicose veins by assisting in moving the vein back to a non-visible location. In some embodiments of the present invention, it may be applied multiple times a day when treating a more extreme case of varicose veins.

The composition of the present invention may comprise a mixture of two solutions: a homogeneous mixture of Glycerin, Caprylic Triglycerides, Oleic Acid, Propylene Glycol, Polysorbate 80 and Isopropyl Alcohol that is mixed with a homogenous mixture of reverse osmosis water, Allantoin, Methyl Nicotinate, Pyridoxal-5-Phosphate (P5P), Phosphatidylcholine, L-Histidine, Glycine, Gingko Biloba Extract, Vitamin K1, Vitamin D3, and Copper Peptides (GHK-Cu). These two homogenous mixtures are mixed well until homogenous to form a liquid.

The liquid, cream, ointment or gel of the present invention may be an oil silicone and water emulsion containing a mixture of protective oils, silicones and waxes. Optionally, a mixture of vitamins, such as vitamins C, E and K, may be included in the cream, gel, ointment, capsule or droplet of the present invention.

As shown in Table 1, the base consists of oils, waters and water-soluble components. In the preferred embodiment, the base is an emulsion of oils, water and water-soluble components. Generally, the base may include any emollients, lubricants, emulsifying agents, thickening agents, humectants, preservatives, fragrances and wetting agents known in the art to be suitable for use in a skin cream base. Also, any mixing methods known in the art to be suitable for mixing an oil and water emulsion for the purposes of forming a skin cream may be used to mix the base ingredients.

The varicose vein diminishing composition should contain a therapeutically effective amount of each of the above described ingredients, and also those described in Table 1. The ingredients of the therapeutic composition of the present invention are summarized in Table 1 below with respect to a description thereof, the active ingredients therein, percentages by weight range for each active ingredient, and the benefits thereof.

TABLE 1

| | Minimum | Maximum | Current | | |
|---|---|---|---|---|---|
| Ingredient | Range % | Range % | % | Description | Benefits |
| Water | 50 | 76 | 61.67 | Water that has | Removes water impurities |
| | | | | undergone a | |
| | | | | water | |
| | | | | purification | |
| | | | | process known | |
| | | | | as reverse | |
| | | | | osmosis, which | |
| | | | | uses a | |
| | | | | semipermeable | |
| | | | | membrane to | |
| | | | | remove ions, | |
| | | | | molecules and | |
| | | | | large particles | |
| | | | | from drinking | |
| | | | | water | |
| Glycerin | 3.5 | 7.5 | 5.5 | Glycerol as is | Moisturizes the skin |
| | | | | generally | |
| | | | | obtained from | |
| | | | | plant and animal | |
| | | | | sources where it | |
| | | | | occurs as | |
| | | | | triglycerides | |
| Caprylic | 3.5 | 7.5 | 5.5 | Medium-chain | Emollient and replenishes |
| Triglycerides | | | | triglycerides who | skin |
| | | | | fatty acids have | |
| | | | | an aliphatic tail | |
| | | | | of 6-12 carbon | |
| | | | | atoms | |
| Oleic Acid | 1.0 | 3.5 | 2.5 | A fatty acid that | Protects cells from free |
| | | | | occurs naturally | radical damage |
| | | | | in various animal | |
| | | | | and vegetable | |
| | | | | fats and oils, | |
| | | | | classified as a | |
| | | | | monounsaturated | |
| | | | | omega-9 fatty | |
| | | | | acid | |
| Propylene | 1.0 | 3.5 | 2.5 | A synthetic | Used to absorb extra |
| Glycol | | | | organic | water and maintain |
| | | | | compound with | moisture |
| | | | | the chemical | |
| | | | | formula | |
| | | | | C3H8O2. | |
| Isopropyl | 1.0 | 2.0 | 1.5 | An isomer of 1- | Helps sooth and reduce |
| Alcohol | | | | propanol that | swelling |
| | | | | contains | |
| | | | | antibacterial | |
| | | | | properties | |
| Polysorbate 80 | .5 | 1.5 | 1.0 | A nonionic | Acts as a surfactant and |
| | | | | surfactant and | emulsifier |
| | | | | emulsifier often | |
| | | | | used in foods | |
| | | | | and cosmetics, | |
| | | | | which is derived | |
| | | | | from | |
| | | | | polyethoxylated | |
| | | | | sorbitan and | |
| | | | | oleic acid | |
| Lecithin | .1 | .5 | .3 | A group of | Used to treat eczema and |
| | | | | yellow-brownish | acts as a moisturizer. |
| | | | | fatty substances | |
| | | | | occurring in | |
| | | | | animal and plant | |
| | | | | tissues, which | |
| | | | | are amphiphilic | |
| Xanthan Gum | .6 | 1.0 | .8 | Polysaccharide | Acts as a thickening agent |
| | | | | used as additive | |
| Allantoin | .2 | .8 | .4 | Chemical | Stimulates cell |
| | | | | compound with | regeneration |
| | | | | formula | |
| | | | | C4H6N4O3, | |
| | | | | which is | |
| | | | | produced from | |
| | | | | uric acid by urate | |
| | | | | oxidase and is | |
| | | | | also a major | |
| | | | | metabolic | |
| | | | | intermediate in | |
| | | | | most organisms, | |
| | | | | including | |
| | | | | animals, plants | |
| | | | | and bacteria | |
| Methyl | .08 | 1.4 | 1.1 | An ester of | Acts as a rubefacient to |
| Nicotinate | | | | methyl alcohol | produce a redness or |
| | | | | and nicotinic | inflammation for a short |
| | | | | acid | period of time |
| Pyridoxal-5- | .1 | .5 | .3 | Active form of | Acts as a coenzyme |
| Phosphate | | | | vitamin B6 and | |
| (P5P) | | | | is a coenzyme in | |
| | | | | a variety of | |
| | | | | enzymatic | |
| | | | | reactions | |
| Phosphatidyl | .05 | .15 | .1 | A class of | Lipid reducer |
| choline | | | | phospholipids | |
| | | | | that incorporate | |
| | | | | choline as a head | |
| | | | | group | |
| L-Histidine | 3.0 | 7.0 | 5.0 | An essential | Used to synthesize |
| | | | | amino acid that | proteins |
| | | | | is one of the 23 | |
| | | | | proteinogenic | |
| | | | | amino acids | |
| Glycine | 3.0 | 7.0 | 5.0 | One of the | Serves as buffering agent |
| | | | | proteinogenic | |
| | | | | amino caids that | |
| | | | | has a single | |
| | | | | hydrogen atom | |
| | | | | as its side chain | |
| | | | | bearing a | |
| | | | | chemical | |
| | | | | formula of NH2— | |
| | | | | CH2—COOH | |
| Gingko Biloba | 1.5 | 4.5 | 3.0 | an extract made | Promotes good blood |
| Extract | | | | from the dried | circulation |
| | | | | green leaves | |
| | | | | from the ginkgo | |
| | | | | trees | |
| Phytonadione | .07 | .09 | .08 | Fat-soluble | Increases the body's |
| | | | | vitamins the | production of blood |
| | | | | human body | clotting factors |
| | | | | requires for | |
| | | | | complete | |
| | | | | synthesis of | |
| | | | | certain proteins | |
| Cholecalciferol | .03 | .07 | .05 | A type of | Helps body absorb |
| | | | | vitamin D made | calcium and phosphorus |
| | | | | in the skin | |
| | | | | following | |
| | | | | ultraviolet-B | |
| | | | | light exposure | |
| Copper | 1.0 | 3.0 | 2.0 | The naturally | Activate the skin's system |
| Peptides (GHK— | | | | occurring copper | responsible for removing |
| Cu) | | | | complex of a | damaged collagen and |
| | | | | glycyl-L- | elastin from the skin |
| | | | | histidyl-L-lysine | |
| | | | | peptide | |
| Sharon ™ | .5 | 1.0 | .8 | Biosecur citrus | Antimicrobial protection |
| Biomix II | | | | extracts with | to cosmetic products |
| | | | | certain fragrance | without using common |
| | | | | ingredients such | preservatives, a |
| | | | | as | combination of Biosecur |
| | | | | Phenylpropanol | citrus extracts with certain |
| | | | | | fragrance ingredients such |
| | | | | | as Phenylpropanol can be |
| | | | | | a good broad spectrum |
| | | | | | solution. |
| Herbal | .5 | 1.5 | 1.0 | NA | Fragrance |
| Fragrance | | | | | |

### Example 1

Composition 1

A therapeutic composition is prepared as described above using the following proportions of ingredients: 61.67% Reverse Osmosis Water, 5.5% Glycerin, 5.5% Caprylic Triglycerides, 2.5% Oleic Acid, 2.5% Propylene Glycol, 1.5% Isopropyl Alcohol (or alternatively Ethanol), 1.0% Polysorbate 80, 0.3% Lecithin, 0.8% Xanthan Gum, 0.4% Allantoin, 1.1% Methyl Nicotinate (Nicotinic Acid), 0.3% Pyridoxal-5-Phosphate (P5P), 0.1% Phosphatidylcholine, 5.0% L-Histidine, 5.0% Glycine, 3.0% Gingko Biloba Extract, 0.08% Phytonadione, 0.05% Cholecalciferol, 2.0% Copper Peptides (GHK-Cu), 0.8% Sharon™ Biomix II, 1.0% Herbal Fragrance.

### Example 2

Evaluation of Test Subjects

A controlled study was performed on the Composition listed in Table 1, by prospectively evaluating its effects on varicose veins through one arm of the study (Study Arm 1) and also through a comparison study by directly comparing the Composition to water-based roll-on liquid (Study Arm 2). In the study, the Composition and the water based substance had similar texture, color and fragrance. In both study arms, the composition and the water were applied twice daily, once in the morning and once in the evening.

The study utilizes “C” or clinical score from the CEAP classification ranges from C0 which means absolutely no venous disease that can be seen or felt in the legs to C6 which means an open and active venous leg ulcer. A full scale is shown in Table 1:

TABLE 1

| CEAP Clinical | |
|---|---|
| Score | Description |
| C0 | No visible or palpable |
| C1 | Telangectasia (Thread veins/Spider veins/Broken veins) |
| C2 | |
| C2A | Varicose veins without any symptoms (Asymtomatic) |
| C2S | Varicose Veins with symptoms |
| C3 | Swollen Ankle (oedema) due to varicose veins or |
| | hidden varicose veins (venous reflux) |
| C4 | Skin damage due to varicose veins or hidden varicose |
| | veins (venous reflux) |
| C5 | Healed venous leg ulcer |
| C6 | Venous leg ulcer |

The study shows a significant improvement in the appearance of the varicose veins. After the test subjects applied the Composition, within ten minutes of application, a reduction of one level occurred (e.g., from C3 to C2 or from C2 to C1). Within forty-five minutes, a significant reduction of varicose veins was observed such that each subject measure a C1 or C0.

There was an overall improvement in the appearance of the veins by 80% at one week, on average, and a two-level decline in C score (e.g., from C3 to C2 or from C2 to C1).

The study also shows that the Composition is superior to a regular water-based substance in all parameters of appearance and patient subject assessment over a 2-month follow-up. More specifically, when comparing the Composition with the water-based substance, an 85% improvement was noted in C-score improvement in the appearance of the varicose veins that used the Composition in comparison to the water-based.

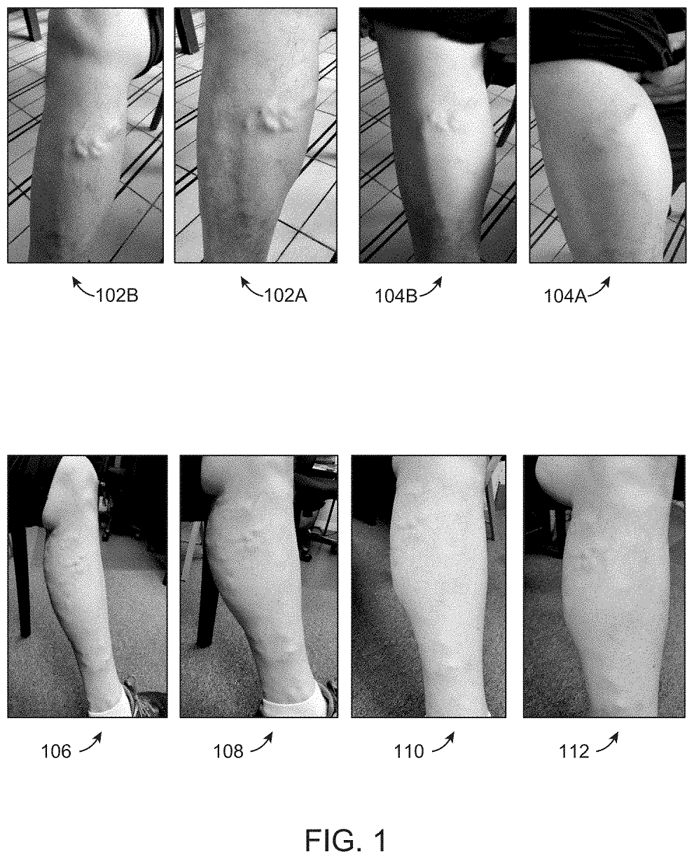

With reference now to FIG. 1, before use of the composition and after use of the composition photographs are shown at 102a and 102b, and 104a and 104b are shown. Further, shown over periods of time after application is photographs 106, 108, 110, and 112, each of which were taken at intervals of 10 minutes, 20 minutes, 30 minutes and 45 minutes respectively.

Application

Beginning one inch above where the bulged vein begins, patients applied the roll-on applicator along the vein until one inch below where it ends. This was repeated three to four times, using fingers to massage vein if necessary. After approximately five minutes, the affected skin felt warm and appear slightly red. This will pass in approximately forty-five minutes, and a significant reduction of varicose veins occurred.

Fifteen minutes after application, the veins appeared significantly reduced in size. If, however, further reduction is desired, patients repeat rolling process, this time allowing forty-five minutes of absorption. Treatment results were complete within two hours of the first application.

Clinical Efficacy

Referring now to Table 2, C-score Scale over time for is presented for Patient average using the Composition:

TABLE 2

| Time | | |
|---|---|---|
| (min) | C-Score | Description |
| 0 | C3 | Swollen Ankle (oedema) due to varicose veins or |
| | | hidden varicose veins (venous reflux) |
| 10 | C2A | Varicose veins without any symptoms |
| | | (Asymtomatic) |
| 20 | C2S | Varicose Veins with symptoms |
| 30 | C1 | Telangectasia (Thread veins/Spider veins/Broken |
| | | veins) |
| 40 | C1 | Telangectasia (Thread veins/Spider veins/Broken |
| | | veins) |
| 50 | C1 | Telangectasia (Thread veins/Spider veins/Broken |
| | | veins) |
| 60 | C0 | No visible or palpable |

Participant Self-Assessment

Participants evaluated their assessment and tolerability of the Compositions using the scale above. The patients applied once in the morning and once in the evening.

Participant Photography

Participants were photographed at each visit using standardized digital photography in the same room using the same lighting conditions and camera settings.

Results

Study Arm 1: Prospective Study Evaluating the Composition

The patients were followed for two months. There was a significant improvement in appearance (See Table 2). During the follow-up period, the participants self-reported significant improvements in the overall appearance and texture of the veins using the composition during the two-month follow-up. There were no issues of tolerability to the composition except for some minor irritation when first applied.

Study Arm 2: Head-to-Head Comparison of the Composition to Water-Based Substance

The patients were followed for two months. There was no improvement in appearance (See Table 2) of varicose veins. me minor irritation when first applied.

General Procedures

The ingredients in the Composition and methods of this invention are either known compounds or are compounds that can be prepared from readily available starting. While optimum reaction conditions may vary with the particular reactants or solvent used, but such conditions can be determined by one skilled in the art by routine optimization procedures.

Topical cosmetic forms and topical pharmaceutical dosing forms can include liquids, creams, gels, ointments, lotions, pastes and the like. The Composition is water based and may use a roll-on application system.

Liquid forms, such as lotions suitable for topical administration as in “drops” may include a suitable aqueous or non-aqueous vehicle with buffers, suspending and dispensing agents, penetration enhancers, and the like.

The above-described components for liquid, semisolid and solid topical compositions are merely representative, and other known topicals may be employed with the present invention.

The following formulation examples illustrate representative pharmaceutical compositions of this invention. The present invention, however, is not limited to the following pharmaceutical compositions.

### Example 3

At room temperature, in the amounts listed in Table 1, Combine: Glycerin, Caprylic Triglycerides, Oleic Acid, Propylene Glycol, Polysorbate 80, and Isopropyl Alcohol. Mix until homogeneous (solution A), then dissolve all other ingredients into the reverse osmosis water until homogeneous (solution B), which may be heated during the mixing process.

Then, solution A with solution B. Mix well until homogeneous. Final solution should be viscous and watery.

In the method of the invention, the composition is administered topically. The method includes the step of topically applying a composition, which includes a therapeutically effective amount of active composition for the treatment of varicose veins, as defined above, and a pharmaceutically acceptable topical carrier, to existing veins. In the method, an effective amount of the topical composition of the invention may be applied to the skin as needed, but preferable, once or twice daily.

More preferably, the topical composition of the present invention is applied to the skin at least once a day, and preferably at least twice (e.g., morning, bedtime) in a 24-hour period.

A topical formulation of the composition used in the invention preferably includes a pharmaceutically acceptable topical carrier. Many pharmaceutically acceptable topical carriers are known to those skilled in the art. The compounds in the composition may be dissolved, dispersed and/or suspended in the topical carrier.

Exemplary topical carriers may include creams, ointments, lotions, pastes, jellies, sprays, aerosols, topical pharmaceutical carriers, which accomplish direct contact between the active ingredients of the topical composition of the present invention and the pore of the skin. One type of pharmaceutically acceptable carrier is a hydrophilic ointment base. Suitable hydrophilic ointment bases are known to persons skilled in the art.

While the present invention has been described in connection with what are presently considered to be the most practical and preferred embodiments, it is to be understood that the present invention is not limited to these herein disclosed embodiments. Rather, the present invention is intended to cover all of the various modifications and equivalent arrangements included within the spirit and scope of the appended claims.

Although specific features of various embodiments of the invention may be shown in some drawings and not in others, this is for convenience only. In accordance with the principles of the invention, the feature(s) of one drawing may be combined with any or all of the features in any of the other drawings. The words “including”, “comprising”, “having”, and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

We claim:

1. A composition for treating varicose veins which comprises 61.67% Reverse Osmosis Water, 5.5% Glycerin, 5.5% Caprylic Triglycerides, 2.5% Oleic Acid, 2.5% Propylene Glycol, 1.5% Isopropyl Alcohol (or alternatively Ethanol), 1.0% Polysorbate 80, 0.3% Lecithin, 0.8% Xanthan Gum, 0.4% Allantoin, 1.1% Methyl Nicotinate (Nicotinic Acid), 0.3% Pyridoxal-5-Phosphate (P5P), 0.1% Phosphatidylcholine, 5.0% L-Histidine, 5.0% Glycine, 3.0% Gingko Biloba Extract, 0.08% Phytonadione, 0.05% Cholecalciferol, 2.0% Copper Peptides (GHK-Cu), 0.8% Sharon™ Biomix II, 1.0% Herbal Fragrance.

2. The composition of claim 1 which comprises 61.67% Reverse Osmosis Water, 5.5% Glycerin, 5.5% Caprylic Triglycerides, 2.5% Oleic Acid, 2.5% Propylene Glycol, 1.5% Isopropyl Alcohol (or alternatively Ethanol), 1.0% Polysorbate 80, 0.3% Lecithin, 0.8% Xanthan Gum, 0.4% Allantoin, 1.1% Methyl Nicotinate (Nicotinic Acid), 0.3% Pyridoxal-5-Phosphate (P5P), 0.1% Phosphatidylcholine, 5.0% L-Histidine, 5.0% Glycine, 3.0% Gingko Biloba Extract, 0.08% Phytonadione, 0.05% Cholecalciferol, 2.0% Copper Peptides (GHK-Cu), 0.8% Sharon™ Biomix II, 1.0% Herbal Fragrance.

3. A method for treating varicose veins, which comprises topically applying to the site of the composition of claim 1.

4. The method of claim 1, wherein varicose veins comprises enlarged and twisted veins.

5. The method of claim 1, wherein varicose veins comprises bulging, blue blood vessels clearly visible through the skin.
